(19) United States
(12) Patent Application Publication
(10) Pub. No.: US 2001/0031284 A1
(43) Pub. Date: Oct. 18, 2001

(54) Process for extracting antioxidants from anaerobically fermented solid plant waste and extracts prepared thereby

(75) Inventors: Jih-Shen Wen, Taipei (TW); Chi-Ming Yang, Taipei (TW)

Correspondence Address: Paul D. Greeley, Esq., Ohland, Greeley, Ruggiero & Perle, Suite903, One Landmark Square, Stamford, CT, 06901, US

(73) Assignee: Jih-Shen Wen, Shu-Linh, Taipei (TW)

(21) Appl. No.: 09/769,178

(22) Filed: Jan. 25, 2001

(30) Foreign Application Priority Data
Mar. 17, 2000 (TW) 089104980

(51) Int. Cl. A61K 35/78; A61K 35/00

(52) U.S. Cl. 424/725; 435/431; 424/115

(57) ABSTRACT

Disclosed is a process for producing antioxidants, which comprises the following steps:

(a) anaerobically fermenting plants;

(b) separating the fermentation broth and collecting the solid residues;

(c) extracting the collected solid residues with a suitable solvent, and collecting the extracts; and

(d) drying the collected extracts.

### FIELD OF THE INVENTION

The invention relates to a process for producing antioxidants. In particular, the invention relates a process for extracting antioxidants from solid waste after anaerobic fermentation of plant.

### BACKGROUND OF THE INVENTION

Increasing the intake of vegetables and fruits is correlated with the prevention of cancers and cardiovascular diseases. It is presumed that the benefit of increasing the intake of vegetables and fruits is associated with the antioxidant ingredient contained therein. Generally, the antioxidation system and the active oxygen in organisms are maintained in equalibrium. When the active oxygen is overly abundant in organisms, a pressure of oxidation is generated and causes a variety of diseases and aging. Vegetables and fruits contain many antioxidants which may neutralize oxygen-containing free radicals. In addition to vitamin C, vitamin E and β-carotene, flavonoids and anthocyanin also exhibit highly antioxidative capability. Therefore, vegetables and fruits are good sources of the combination of natural antioxidants.

Statistics show that at least 90 tons of the waste of vegetables and fruits are produced per year in Taiwan and create serious environmental problems. Therefore, reduction and recycle of the waste of vegetables and fruits are of great urgency.

At present, treatment of the waste of vegetables and fruits is mainly to bury them. Treatment of the waste of vegetables and fruits by anaerobic fermentation is also utilized. While the marsh gas produced during the fermentation is used as gas fuel after a subsequent deodorization and purification, the solid and liquid residue are used as natural organic fertilizers.

However, no publication or patent discloses the utilization of the waste of vegetables and fruits in the preparation of antioxidants.

### SUMMARY OF THE INVENTION

The object of the invention is to provide a process for producing antioxidants, which comprises the treatment of anaerobic fermentation and is suitable for all plant materials, including the waste of vegetables, and fruits, and herbals.

Another object of the invention is to provide an antioxidant mixture prepared according to the process of the invention.

### DETAILED DESCRIPTION OF THE INVENTION

The invention in the first aspect provides a process for producing antioxidants, comprising the following steps:

(a) anaerobically fermenting plants;

(b) separating the fermentation broth, and collecting the solid residues;

(c) extracting the collected solid residues with a suitable solvent, and collecting the extracts; and

(d) drying the collected extracts.

Plant materials suitable for the anaerobic fermentation step of the invention can be any species of plants or the parts thereof, in particular those known to contain antioxidants, such as carrot, tomato or herbals used in Chinese herbal medicine. Vegetables, fruits or herbals are the preferable plant sources. More preferably the sources are the waste of vegetables and fruits such that the purpose of waste recycling may be achieved.

The anaerobic fermentation in step (a) can be performed according to any manner known to persons skilled in the art. For instance, the anaerobic fermentation can be performed at the temperature of 20 to 60° C., preferably at the temperature of 35 or 55° C., for 3 to 4 days. The fermentation is preferably conducted in the manner of a two-stage anaerobic fermentation. The first stage is to ferment in a closed tank, and the second stage is to anaerobically ferment in a methylation tank. For instance, the methylation can be performed in a methylation tank containing a mixed-bed tower reactor filled with polyethylene glycol polymerized supports.

After the fermentation, the resultant residues are separated into solid residues and liquids. In general, the separation of solid residues and liquids in step (b) can be performed by any method known to persons skilled in the art, which includes, but is not limited to, centrifugation and collection of the residues.

The extraction in step (c) can be performed with any suitable organic solvents and/or water. The organic solvent is preferably methanol, ethanol, n-hexane or the mixtures thereof.

The drying in step (d) can be performed by any methods known to persons skilled in the art, which includes, but is not limited to, freeze-drying, spray-drying, drum drying or fluid-bed drying, preferably freeze-drying.

The extracts obtained according to the invention exhibit significant in the low-density lipoprotein system. Therefore, the invention further provides an antioxidant mixture prepared by the process of the invention.

The antioxidative capability of the antioxidant mixtures prepared by the process of the invention can be determined by any tests known to persons skilled in the art, such as the low-density lipoprotein oxidation sensitivity test, the oleic acid anti-oxidation method, the reduction power test, the scavenging of free radical of α,α-diphenyl-β-phinohydzaine (DPPH) test, the ferric ion chelating test and the elimination of ability of superoxide ion test.

The antioxidant mixture prepared according to the invention can be used in feeds, foods, cosmetics, pharmaceuticals, preservatives, organic fertilizers or seed-immersing liquids.

The following examples further illustrate the invention but are not intended to limit the invention. Any modification and application obvious to persons skilled in the art in light of the teachings of the invention is contemplated to be within the scope of the invention.

### EXAMPLE

### Example 1

Anaerobic Fermentation of the Waste of Vegetables and Fruits

(1) Treatment of Vegetable and Fruit Materials

The waste of vegetables and fruits collected in the market or farmwas filled into a plastic barrel. Bamboo rods with a sharp top were used to puncture the waste for about 150 times.

(2) Bacteria Source

Soil obtained from the anaerobic pool of pigpen was sieved with 2 and 30-mesh filters to remove gravels. Calcium acetate (1000 ppm) was added thereto for flocculation. The flocculated bacteria slurry was used as the inoculation source.

(3) Cultivation by Anaerobic Fermentation

The bacteria slurry was inoculated into the barrel. The barrel was tightly sealed with a cover and an anaerobic fermentation was performed at 35 or 55° C. for 3 to 4 days.

(4) Methylation of Anaerobically Fermented Waste

Solid and liquid residues obtained from the process of anaerobic fermentation were transferred into a methylation tank containing a mixed-bed tower reactor filled with 3 cm³ of polyethylene glycol polymerized supports and a methylation was conducted at 35 or 55° C. for 3 to 4 days. Anaerobically fermented waste of vegetables and fruits was obtained.

### Example 2

Extraction of Antioxidants

The solid residues of vegetables and fruits obtained from the anaerobic fermentation were centrifuged (4,500 rpm, 4° C.) for 15 minutes. The lower layer of the precipitates (solid residues) were collected, poured into small vessels, transferred to a freezer of −70° C., and frozen overnight. The precipitates were then filled into lyophilization vessels and lyophilized under a reduced pressure for 1 to 2 days. The dried solid residues in the vessels were mixed and grinded to fine powders by use of liquid nitrogen, and then poured into small vessels and stored in a freezer of −20° C. The solid residue powder was extracted with methanol, n-hexane, ethanol or an aqueous solution, respectively. The extracts obtained from the former three organic solvents were concentrated under a reduced pressure or dried with nitrogen gas. The extract obtained form the aqueous solution was lyophilized to obtain the dried extract.

(1) Extraction of Organic Phase

Solid residues (1 g) were extracted with 10 ml ethanol, methanol or n-hexane, respectively, and homogenized with a homogenizer. They were centrifuged at 2,000 g under 4° C. for 10 minutes, and the supernatant of the liquids was collected. The above procedures were repeated 5 times until the extracts became colorless. The supernatant was combined, and the organic phase was collected and then concentrated under a reduced pressure at ambient temperature or dried with nitrogen gas. Finally, the supernatant was dehydrated in a lyophilization drier. The dehydrates were stored at −20° C. before use.

(2) Extraction of Water Phase

Solid residues (1 g) were extracted with 5 ml of a 10 mM Tris solution (pH 7.0) and ethylene polyethylenepyrrolidone, and homogenized with a homogenizer, followed by a centrifugations at 25,000 g under 4° C. for 30 minutes. The supernatant of the liquid was collected. The above procedures were repeated 6 times until the extracts became colorless. The supernatant of all aqueous phases was combined and dehydrated in a lyophilization drier. The dehydrates were stored at −20° C. before use.

### Example 3

Determination of Oxidation Ability Sensitivity in Low-Density Lipoprotein System

(1) Purification of Low-Density Lipoprotein

Thirty ml of venous blood was injected to a blood collecting vessel containing 1.5 mg/ml EDTA-K3, and then centrifuged at 3,000 rpm under 4° C. for 15 minutes to obtain blood plasma. Then, the blood plasma was subject to ultra-centrifugation (1.006<d<1.163 g/ml) to seperate the low-density lipoprotein. First, the very low-density lipoproteion (VLDL) layer was removed by centrifugation at 44,000 rpm for 16 hours (d>1.006 g/ml), and then for additional 20 hours to obtain the low-density lipoprotein (LDL) layer. The low-density lipoprotein was subject to dialysis with a 50 mM phosphate buffer (pH 7.4) for 22 hours. Then the low-density lipoprotein was used to determine the peroxidation sensitivity.

(2) Determination of Peroxidation Sensitivity of Low-Density Lipoprotein

The dialyzed low-density lipoprotein was diluted to a concentration of 0.9 mg/ml of cholesterol. A certain amount of low-density lipoprotein and 25 μM CuSO₄ were incubated at 37° C. for 5 hours. The amount of malonyldialdehyde (MDA) produced at the time intervals of 0, 30, 60, 70, 80, 90, 100, 120, 150, 180, 240 and 300 minutes, respectively, was measured. One hundred μL of trichloroacetic acid (15.2%, w/v) was added to terminate the reaction. A certain amount of the supernatant was added with 1 μL of trifluoroacetic acid (0.6%, w/v) incubated at 100° C. for 30 minutes and then cooled in cold water. The absorption of the supernatant at 532 nm was determined. The amount of MDA produced was calculated accordingly.

After the amount of MDA produced was converted, the antioxidative capability of different extracts in the low-density lipoprotein system was obtained. The results are shown as 150 value as follows:

| Extraction phase | I₅₀ (mg/ml) |
|---|---|
| Aqueous Solution | 130 |
| Ethanol | 28 |
| Methanol | 13 |
| n-Hexane | 24 |

The value of I₅₀ shown in the table represents the concentration of the extract required for the inhibition of 50% LDL (i.e., MDA produced). From the results of the table, it is known that the mixture extracted with methanol exhibits the best antioxidative capability.

We claim:

1. A process for producing antioxidants, which comprises the following step:
(a) anaerobically fermenting plants;
(b) separating the fermentation broth, and collecting the solid residues;
(c) extracting the collected solid residues with a suitable solvent, and collecting the extracts; and
(d) drying the collected extracts.

2. The process according to claim 1, wherein the plants are vegetables and fruits.

3. The process according to claim 2, wherein the vegetables and fruits are the waste of vegetables and fruits.

4. The process according to claim 1, wherein the plants are herbals.

5. The process according to claim 1, wherein the fermentation is a two-stage anaerobic fermentation, the first stage of which is to ferment in a closed tank, and the second stage of which is to perform an anaerobic fermentation in a methylation tank.

6. The process according to claim 5, wherein the methylation tank contains a mixed-bed tower reactor filled with polyethylene glycol polymerized supports.

7. The process according to claim 1, wherein the solvent is an organic solvent.

8. The process according to claim 7, wherein the organic solvent is selected from the group consisting of methanol, ethanol and n-hexane.

9. The process according to claim 8, wherein the organic solvent is methanol.

10. The process according to claim 1, wherein the solvent is water.

11. The process according to claim 1, wherein the drying is freeze-drying.

12. The process according to claim 1, wherein the temperature of the anaerobic fermentation is from 20 to 60° C.

13. The process according to claim 12, wherein the temperature of the anaerobic fermentation is 35° C.

14. The process according to claim 12, wherein the temperature of the anaerobic fermentation is 55° C.

15. An antioxidant mixture prepared by the process according to claim 1.

16. The mixture according to claim 15, which is used in feeds, foods, cosmetics, pharmaceuticals, preservatives, organic fertilizers and seed-immersing liquids.
